(19) United States
(12) Patent Application Publication
(10) Pub. No.: US 2018/0014937 A1
(43) Pub. Date: Jan. 18, 2018

(54) NITINOL METAL INJECTION MOLDING OF POROUS, ORTHOPEDIC IMPLANTS WITH A TITANIUM SUBSTRATE

(71) Applicant: Arthrex, Inc., Naples, FL (US)

(72) Inventors: Matthew Fonte, Concord, MA (US); Matthew Palmer, Medford, CA (US); Robert Devaney, Auburndale, MA (US)

(21) Appl. No.: 15/647,799

(22) Filed: Jul. 12, 2017

Related U.S. Application Data
(60) Provisional application No. 62/361,275, filed on Jul. 12, 2016.

(51) Int. Cl. A61F 2/30

(52) U.S. Cl. CPC A61F 2/30; A61F 2310/00461; A61F 2310/00407; A61F 2310/00023; A61F 2002/30971; A61F 2002/30092; A61F 2002/30784; A61F 2002/30738; A61F 2002/30317; A61F 2002/30141; A61F 2002/3092

(57) ABSTRACT

In one example, a method for providing therapy to a patient includes inserting a medical implant into the patient, where the medical implant comprises a titanium substrate metallurgically bonded to a dynamic porous material comprising a shape memory alloy. The dynamic porous material conforms to an adjacent bone to create an interference fit between the medical implant and the adjacent bone.

REFERENCE TO PENDING PRIOR PATENT APPLICATION

This patent application claims priority to and the benefit of pending prior U.S. Provisional Patent Application Ser. No. 62/361,275, filed on Jul. 12, 2016, the entire disclosure of which is hereby incorporated by reference.

### FIELD OF THE INVENTION

A purpose of the present invention is to improve bone ingrowth into orthopedic implants by creating an implant with a dynamic surface coating. The dynamic surface coating is less stiff to address stress shielding and superelastically conforms to bone after implantation to improve fixation (for the purposes of the present invention, the term “dynamic” may be considered to mean capable of changing shape, e.g., due to structural resilience, including superelasticity, thermal shape recovery, etc.). The dynamic surface coating consists of a shape memory material, e.g., Nitinol, made with a three dimensional porous structure. The pores of the dynamic surface coatings can be infiltrated with a mixture of hydroxyapatite, tricalcium phosphate, and other bone-promoting agents known in the art. This invention finds utility as a dynamic surface coating in many orthopedic implants where fixation and osseointegration are essential, e.g., knee implants, shoulder implants, elbow implants, spinal implants, maxillofacial implants, cranial implants, extremity (e.g., fingers and toes) implants, etc.

### BACKGROUND

Metal injection molding (MIM) is a metalworking process by which finely-powdered metal is mixed with a measured amount of binder material to comprise a “feedstock” capable of being handled by plastic processing equipment through a process known as injection mold forming. The molding process allows complex parts to be shaped in a single operation and in high volume. The end products are commonly component items used in various industries and applications. The nature of metal injection molding feedstock flow is defined by a physics called rheology. Current equipment capability requires processing to stay limited to products that can be molded using typical volumes of 100 grams or less per “shot” into the mold. Rheology does allow this “shot” to be distributed into multiple cavities, thus becoming cost-effective for small, intricate, high-volume products which would otherwise be quite expensive to produce by alternate or classic methods. The variety of metals capable of implementation within metal injection molded feedstock are referred to as powder metallurgy, and these contain the same alloying constituents found in industry standards for common and exotic metal applications. Subsequent conditioning operations are performed on the molded shape where the binder material is removed, and the metal particles are coalesced into the desired state for the metal alloy.

The process steps involve combining metal powders with wax and plastic binders to produce the “feedstock” mix that is injected as a liquid into a hollow mold using plastic injection molding machines. The “green part” is cooled and de-molded in the plastic molding machine. Next, a portion of the binder material is removed using solvent, thermal furnaces, catalytic process, or a combination of methods. The resulting, fragile and porous (2-4% “air”) part, in a condition called “brown” stage, requires the metal to be condensed in a furnace process called Sintering. Metal injection molded parts are sintered at temperatures nearly high enough to melt the entire metal part outright (up to 1450 degrees Celsius), at which the metal particle surfaces bind together to result in a final 96-99% solid density. The end-product metal injection molded metal has comparable mechanical and physical properties with parts made using classic metalworking methods, and metal injection molded materials are compatible with the same subsequent metal conditioning treatments, such as plating, passivating, annealing, carburizing, nitriding, and precipitation hardening.

In addition to the foregoing, and as noted above, porous metals have not only been used as coatings on implants, but have also been used as the implant itself. Porous metal implants have been introduced into a void in bone to act as bone void fillers and bone augmentation devices. These porous metal implants are often used as part of a revision procedure after the primary implant has failed and has been removed. Removal of the implant leaves behind a void in the bone. Instead of filling the void with bone cements, it has become popular to implant a porous metal implant to fill some of the space in the bone and to provide support for the revision implant. Bone thereafter grows into the porous metal implant, securely holding the porous metal implant in place, typically adjacent to the revision implant.

Porous metal implants have also been used as wedges for osteotomies. In these procedures, an opening wedge osteotomy is performed, and the porous metal implant is used to keep the osteotomy open. The porous metal composition of the wedge allows for bone ingrowth and eventual fusion of the osteotomy site.

Current porous metal implants are static and are unable to conform to the geometry of the anatomy. Thus, it has been observed that gaps exist between the implants and the bone. This greatly impairs the bone's ability to grow into the porous metal scaffold. This is especially true for porous metal implants used as part of a revision procedure. Removal of a primary implant often creates an irregularly-shaped bone void which is difficult to completely fill using a conventional porous metal implant.

Thus, there exists a clinical need for dynamic porous metal implants that are able to conform to implantation sites so as to maximize the surface area of implant-bone contact. These dynamic porous metal implants may also be less stiff to address stress shielding. Also, they may be elastic and attempt to expand after implantation to improve implant fixation and to apply stress to the adjacent bone so as to enhance bone remodeling and ingrowth.

### SUMMARY

In one example, a method for providing therapy to a patient includes inserting a medical implant into the patient, where the medical implant comprises a titanium substrate metallurgically bonded to a dynamic porous material comprising a shape memory alloy. The dynamic porous material conforms to an adjacent bone to create an interference fit between the medical implant and the adjacent bone.

In another embodiment according to any of the previous embodiments, the medical implant includes one of a bone void filler and a bone augmentation device.

In another embodiment according to any of the previous embodiments, the porous structure has one of a dodecahedron cellular structure or a singular structure.

In another embodiment according to any of the previous embodiments, the medical implant includes a non-dynamic core and a dynamic surface.

In another embodiment according to any of the previous embodiments, the porous structure has the singular structure having a structural or material gradient.

In another example, a medical implant includes a titanium substrate bonded to a dynamic porous structure comprising a dynamic porous material comprising a shape memory material.

In another embodiment according to any of the previous embodiments, the shape memory material is Nitinol.

In another embodiment according to any of the previous embodiments, the medical implant is expandable to fill gaps between the medical implant and adjacent bone when implanted into a patient.

In another embodiment according to any of the previous embodiments, the dynamic porous structure is metal injection molded device.

In another embodiment according to any of the previous embodiments, the dynamic porous structure includes a singular structure.

In another embodiment according to any of the previous embodiments, the medical implant has a non-dynamic core and a dynamic surface.

In another embodiment according to any of the previous embodiments, the dynamic porous structure comprises a dodecahedron cellular structure.

In another example, a porous orthopedic implant includes a titanium alloy substrate and a porous portion including a mixture of a material blended with a coated pore former and a homogenizing agent. The porous portion includes a Nitinol alloy.

In another embodiment according to any of the previous embodiments, the porous portion includes pores having a diameter of about 50 microns to about 2,000 microns.

In another embodiment according to any of the previous embodiments, the porous portion is about 60% to about 85% porous.

These and other features of the present invention can be best understood from the following specification and drawings, the following of which is a brief description.

### BRIEF DESCRIPTION OF THE DRAWINGS

FIG. 1 shows a medical implant including a titanium substrate and a dynamic porous material;

FIG. 2 shows a component having a complicated internal geometry formed by an injection molding process;

FIG. 3 shows a close up view of the component;

FIG. 4 shows a graph of increased oxygen uptake with debinding temperature for NiTi powder held at various temperatures;

FIG. 5 shows data regarding different sized Nitinol powders;

FIG. 6 shows pores that remain after sintering; and

FIG. 7 shows a chart summarizing Nitinol metal injection molding of porous, superelastic implants with a titanium substrate.

### DETAILED DESCRIPTION

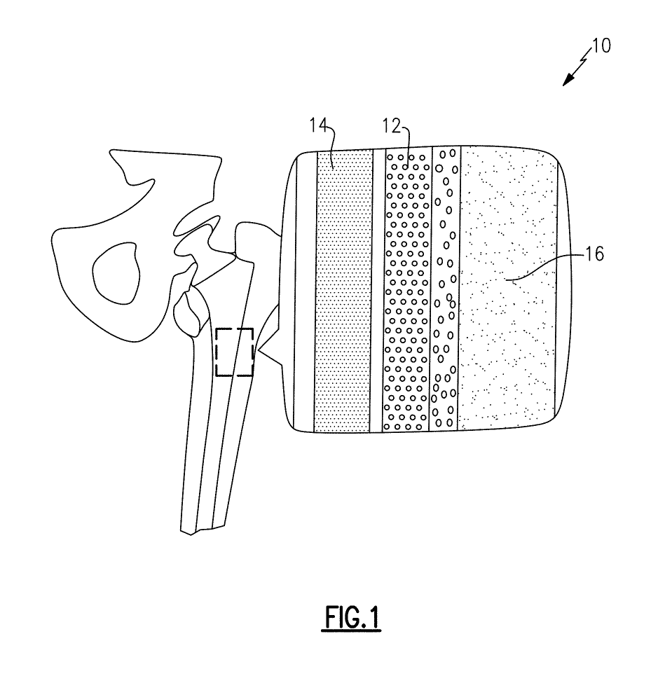

As shown in FIG. 1, the present invention provides a novel dynamic porous medical implant 10 that consists of a shape memory material, e.g., Nitinol, three dimensional porous structure 12 with a metal substrate 14 made by metal injection molding. The substrate core 14 could be made of cobalt chrome, stainless or titanium alloy. Pores can be infiltrated with a mixture of hydroxyapatite, tricalcium phosphate, and other bone-promoting agents known in the art. This invention finds utility as a dynamic porous implant where fixation and osseointegration are essential, e.g., bone void fillers, cement spacers, femoral and tibial cone augments, buttresses, cages and other bone augmentation devices, including bone wedges, such as cotton and evans wedges.

In one preferred form of the invention, there is provided a porous coating 12 for a medical implant 10. The porous coating 12 comprises a porous, shape memory material.

In another preferred form of the invention, there is provided a medical implant 10 including a body 14 and a porous coating 12 secured to a surface of the body 14, and the porous coating 12 comprises a porous, shape memory material.

In another preferred form of the invention, there is provided a method for providing therapy to a patient. The method includes providing a medical implant 10 including a body 14 and a porous coating 12 secured to a surface of the body 14. The porous coating 12 includes a porous, shape memory material. The method includes inserting the medical implant 10 into the patient so the porous coating 12 applies an outward force against adjacent bone 16 to fill gaps between the porous coating 12 and the adjacent bone 16 and to create an interference fit between the medical implant 10 and the adjacent bone 16.

In one example, the coating 12 on the medical implant 10 has a modulus between 1-20 GPa. The porous structure has one of a dodecahedron cellular structure or a singular structure. The medical implant 10 includes a non-dynamic core and a dynamic surface. If the porous structure has a singular structure, it has a structural or material gradient.

The medical implant 10 can include a titanium alloy substrate 14 and a porous portion 12 including a mixture of a material blended with a coated pore former and a homogenizing agent, and the porous portion is a Nitinol alloy. The porous portion can include pores having a diameter between about 50 microns and about 2,000 microns. The porous portion can be between about 60% and about 85% porous.

A porous article for use as a biological implant includes a porous portion of open cell porosity with a substantially uniform interconnecting pore ratio of major pore size to minor pore size, and the porous portion is Nitinol and superelastic. The interconnecting pore ratio is between about 2:1 and about 5:1 of major pore size to minor pore size. The porous portion is a first portion to support bone ingrowth, and the porous article has a second portion of negligible porosity to avoid bone ingrowth.

In another preferred form of the invention, there is provided a method for providing therapy to a patient, the method comprising providing a medical implant 10 comprising a dynamic porous material 12. The dynamic porous material 12 comprises a porous structure formed of a shape memory material, and the porous structure comprises a regular repeating pattern. The method includes providing a medical implant 10 comprising a dynamic porous material. The dynamic porous material comprises a porous structure formed of a shape memory material, and the porous structure comprises a non-regular repeating pattern. The method includes inserting the medical implant 10 into a patient so that the dynamic porous implant 10 applies an outward force against adjacent bone 16 to create an interference fit between the medical implant 10 and the adjacent bone 16.

In another preferred form of the invention, there is provided a medical implant 10 comprising a dynamic porous material 12. The dynamic porous material 12 comprises a porous structure formed of a shape memory material, and the porous structure comprises a regular repeating pattern or a non-repeating (random pattern). The medical implant 10 is configured for insertion into a patient so that the dynamic porous implant 10 applies an outward force against adjacent bone 16 to create an interference fit between the medical implant 10 and the adjacent bone 16.

Nitinol MIM (metal injection molding) allows parts to be manufactured to the ASTM F2885 Standard Specification for metal injection molded Titanium-6Aluminum-4Vanadium Components for Surgical Implant Applications, but made out of Nitinol, not Ti 6A1-4V. Fatigue performance in excess of 70-90 ksi at 10 million cycles is at risk of rupture in flexural bending (RBF). This allows the metal injection molding to be used in load-bearing or fatigue-sensitive applications, such as orthopedic implants.

The Process

1. Feedstock Formation: Fine nickel and titanium powder metal, or Nitinol powder, are combined with thermoplastic binders at precise levels. The materials are mixed together and heated up to allow the metal powders to disperse within the melted binders. The mixture is then pelletized to form a feedstock suitable for injection molding.

2. Molding: Metal injection molding, like plastic injection molding, uses a conventional injection molding machine to form a molded part. In the case of metal injection molding, feedstock is fed from a hopper into a heated barrel, where the feedstock is melted. However, only the binders are melted. Once the feedstock is molten, it is injected into a mold to form a desired geometry. Once the part is cool, the part is ejected and ready for debinding. At this point, the molded part is referred to as a “green part.”

3. Debinding: The debinding process removes only a portion of the binder components. The remaining binder will stay to hold the part together during the first part of sintering. Debinding can be carried out in multiple ways, the most common routes are solvent extraction or catalytic decomposition.

4. Sintering/Thermal Processing: Debound parts are placed on ceramic setters and loaded into a furnace for high temperature processing. During the early stage of sintering, the remaining binder is thermally decomposed. After this initial stage, the parts are heated to a high temperature where densification occurs, resulting is significant shrinkage of up to 20%.

5. Resulting Solid Component: The resulting solid component is nearly 100% dense and identical in chemistry to conventional titanium.

6. HIP'ing/Secondary Operations: To achieve full density, the component may be hot isostatically pressed (HIP'd). Secondary finishing options, such as CNC machining, anodizing, passivation, surface finishing, and laser marking are also possible.

A co-forming technique developed for orthopedic implants produces a tailorable Nitinol surface to a titanium injection molded component in one step. An additively manufactured sacrificial insert is placed into the mold to simultaneously co-form the porous Nitinol surface to the solid titanium component. This innovative approach allows net shape forming of the Nitinol porous surface texture and solid titanium part in one molding process. After molding, the sacrificial insert is removed, and the product is processed similarly to a conventional metal injection molding process. This surface can be engineered to be an ingrowth, ongrowth, or polymer anchoring surface. Since the entire surface is defined by the sacrificial insert, the nature of the surface texture, the porous section, and the interface between the porous and solid sections can be controlled.

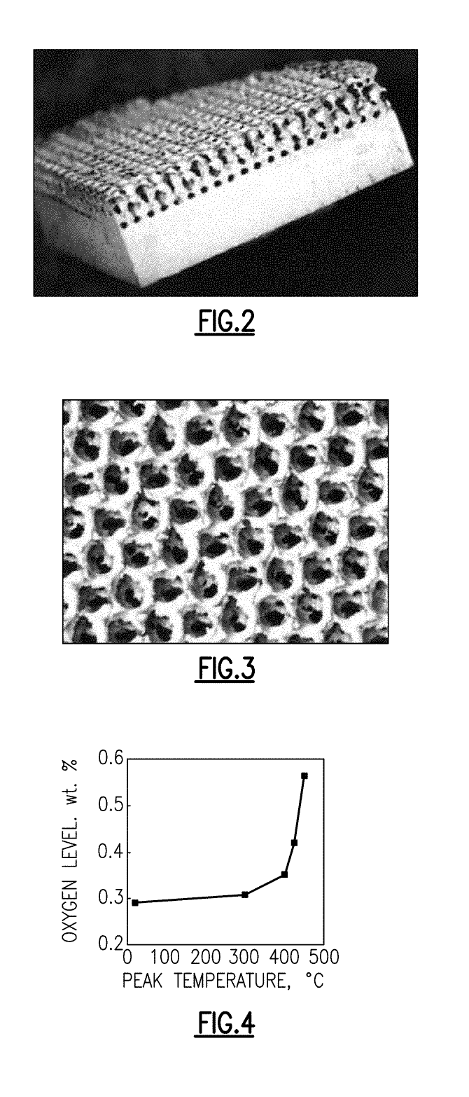

As shown in FIGS. 2 and 3, by incorporating sacrificial inserts onto a component manufactured by Nitinol injection molding, complicated internal geometries or undercuts are easily formed. Sacrificial inserts make the injection molding process even more flexible from a design standpoint. Geometries that would be considered impossible or prohibitively expensive can be molded into the part in a net shape fashion. This allows increased complexity and decreased weight in a variety of components. By using an additive manufacturing process, sacrificial negatives of an integration surface are created. As the feedstock is forced into the mold, it flows into the insert. The insert is subsequently removed, leaving behind a network of interconnected passages with precise pore sizes and porosity. Sacrificial inserts can be used to create hollow sections within a molded part for the purpose of decreasing weight or creating complicated geometries. Device surface scaffolds should have an interconnected pore size between 100-500 micrometers.

A cold isostatic pressing step between debinding and sintering is recommended. Using spherical gas atomized −325 mesh (−45 μm) Nitinol powder (0.008% C and 0.140% 0), the powders are premixed, and then feedstock is mixed at 173° C. for 2.5 hours to give 65 volume % solids loading. After molding, the wax phase is removed via heptane immersion for 6 hours, followed by vacuum sweep-gas treatment at 430° C. Sintering densification is at 1350° C. for 4 hours. The sintered density is higher using pre-alloyed powders, exceeding 97% fractional density, with a tensile strength of 830 MPa with 0.31% oxygen.

Use of mixed atomized and hydride-milled-dehydride (HDH) powders provides a means to control rheology and cost. Binder design needs to balance powder wetting, rheology, green strength, debinding, and contamination concerns. Mixing of the nickel and titanium powder, or Nitinol powder, and binder at low temperatures prevents oxidation, and mixing under inert gas is generally most beneficial. Debinding is a sensitive aspect of NiTi-MIM (metal injection molding) and requires two steps: solvent immersion followed by thermal pyrolysis under vacuum using the sweep-gas concept. The peak temperature, hold time, and other parameters are determined using analytical tools, including mass spectroscopy or similar in situ monitors. FIG. 4 shows a graph of the increased oxygen uptake with debinding temperature for NiTi powder held at various temperatures.

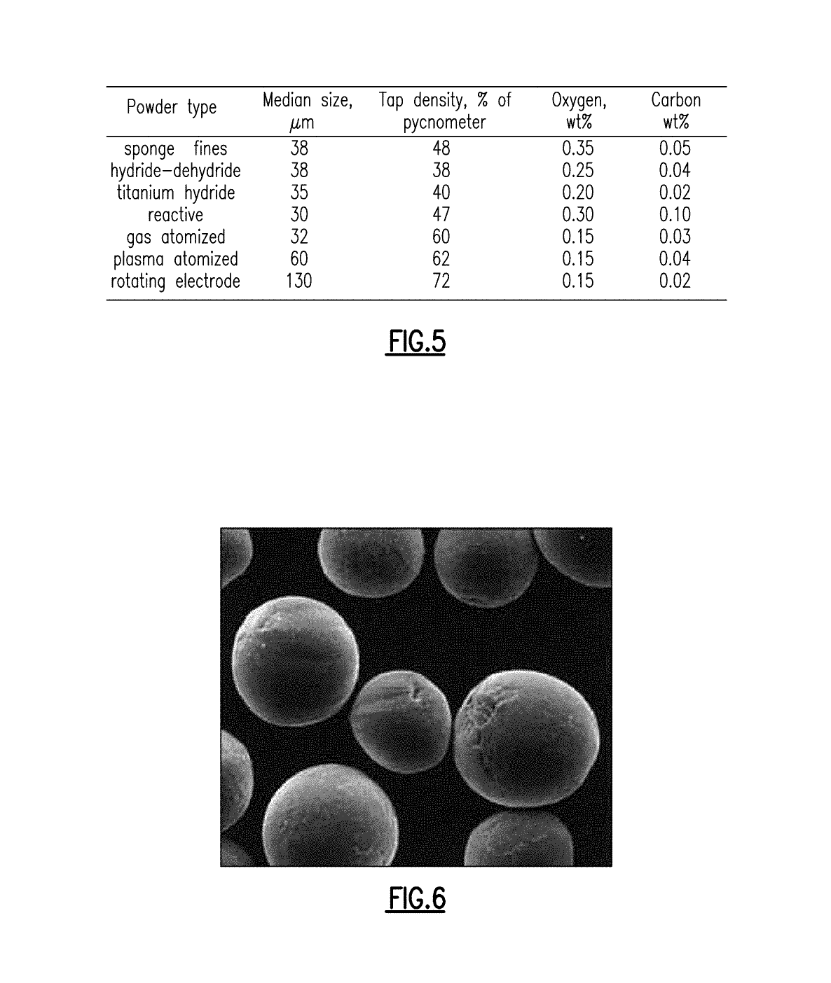

To minimize impurities, Nitinol powder selection favors a large particle size with less surface area to limit reactions. As show in FIG. 5, different particle sizes of Nitinol powder use a wax-polymer binder and two-step (solvent and thermal) debinding, followed by vacuum sintering at 1,350 degrees Celsius for 90 minutes. The smaller particle size requirement provide a higher molding pressure, slower debinding, and results in higher sintered density. The impurity level after sintering is higher.

A typical compromise for Nitinol powder is to use −325 mesh (below 45 μm) spherical or tumbled powder customized to give a high tap density. The starting oxygen level is below 0.20 weight %, and the carbon level is below 0.05 weight %. One trick is to use hydride powders, where the hydrogen liberated during sintering helps remove volatile impurities, especially those arising from the sintering atmosphere. The need for the post-sintering hot isostatically pressed treatment is evident by the sintered microstructure. As shown in FIG. 6, the black spots are pores that remain after sintering. Note the large grain size indicative of considerable coarsening prior to full densification.

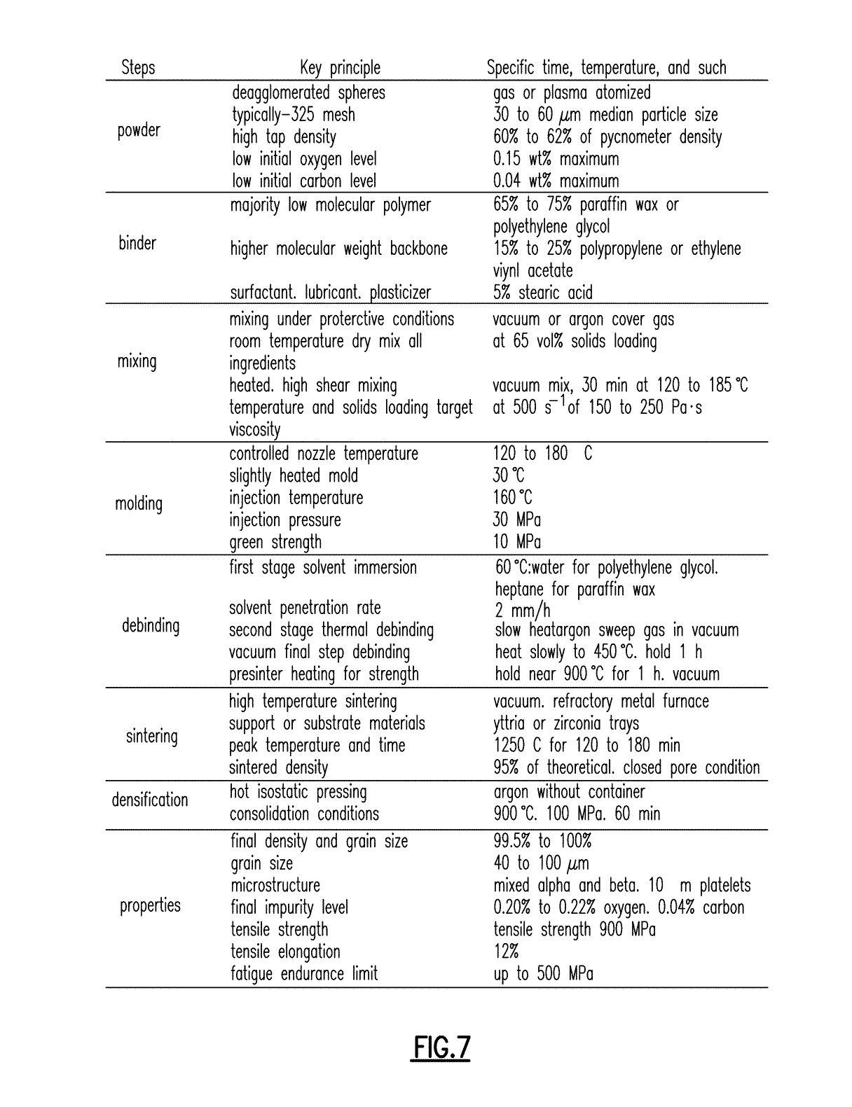

In this case, the sintering temperature results in a mixture of two phases, giving a desirable lamellar structure. The grains are over 100 μm in size, the pores are about 10 to 15 μm in diameter, and the lamellar plates are about 5 to 10 μm. Because of the porosity and coarse microstructure, the mechanical properties are typically the same as for cast material. After the hot isostatically pressed treatment, the grain size is similar, but the pores are absent, and no artifact of the powder process remains. FIG. 7 shows a chart summarizing Nitinol Metal Injection Molding of Porous, Superelastic Implants with a titanium substrate.

A method of producing an orthopedic implant with a dynamic porous surface includes blending gas or atomized powder of titanium and nickel alloy that has a particle size between 30-60 μm and oxygen at 0.15% weight maximum. The method includes mixing in a binder under vacuum or in argon or nitrogen gas, with at least 65% paraffin wax or polyethylene glycol and 5% stearic acid. The method also includes injection molding between 120° C. to 180° C. at an injection pressure temperature between 10 MPa and 40 MPa. The method also includes debinding in water or polyethylene glycol or heptane and heating in vacuum to between 400° C.-600° C. for 1 hour near 900° C. The method includes sintering the titanium implant substrate between 1,000° C.-1,275° C. for 1 hour and 1.5 hours. The method finally includes densifying in argon or inert atmosphere near 900° C. between 50 MPa-150 MPa for between 30-90 minutes.

The final density is equal to or greater than 98%, has a grain size of 40 to 100 μm and has a superelasticity. The conforming porous coating is created by the superelasticity of the shape memory material, and the substrate implant is created by titanium or cobalt chrome.

In one example, a method for providing therapy to a patient includes inserting a medical implant into the patient, where the medical implant comprises a titanium substrate metallurgically bonded to a dynamic porous material comprising a shape memory alloy. The dynamic porous material conforms to an adjacent bone to create an interference fit between the medical implant and the adjacent bone.

In another embodiment according to any of the previous embodiments, the medical implant includes one of a bone void filler and a bone augmentation device.

In another embodiment according to any of the previous embodiments, the porous structure has one of a dodecahedron cellular structure or a singular structure.

In another embodiment according to any of the previous embodiments, the medical implant includes a non-dynamic core and a dynamic surface.

In another embodiment according to any of the previous embodiments, the porous structure has the singular structure having a structural or material gradient.

In another example, a medical implant includes a titanium substrate bonded to a dynamic porous structure comprising a dynamic porous material comprising a shape memory material.

In another embodiment according to any of the previous embodiments, the shape memory material is Nitinol.

In another embodiment according to any of the previous embodiments, the medical implant is expandable to fill gaps between the medical implant and adjacent bone when implanted into a patient.

In another embodiment according to any of the previous embodiments, the dynamic porous structure is metal injection molded device.

In another embodiment according to any of the previous embodiments, the dynamic porous structure includes a singular structure.

In another embodiment according to any of the previous embodiments, the medical implant has a non-dynamic core and a dynamic surface.

In another embodiment according to any of the previous embodiments, the dynamic porous structure comprises a dodecahedron cellular structure.

In another example, a porous orthopedic implant includes a titanium alloy substrate and a porous portion including a mixture of a material blended with a coated pore former and a homogenizing agent. The porous portion includes a Nitinol alloy.

In another embodiment according to any of the previous embodiments, the porous portion includes pores having a diameter of about 50 microns to about 2,000 microns.

In another embodiment according to any of the previous embodiments, the porous portion is about 60% to about 85% porous.

Although a preferred embodiment of this invention has been disclosed, a worker of ordinary skill in this art would recognize that certain modifications would come within the scope of this invention. For that reason, the following claims should be studied to determine the true scope and content of this invention.

What is claimed is:

1. A method for providing therapy to a patient, the method comprising:
inserting a medical implant into the patient,
wherein the medical implant comprises a titanium substrate metallurgically bonded to a dynamic porous material comprising a shape memory alloy,
wherein the dynamic porous material conforms to an adjacent bone to create an interference fit between the medical implant and the adjacent bone.

2. The method as recited in claim 1 wherein the medical implant includes one of a bone void filler and a bone augmentation device.

3. The method as recited in claim 1 wherein the porous structure has one of a dodecahedron cellular structure or a singular structure.

4. The method as recited in claim 3 wherein the medical implant includes a non-dynamic core and a dynamic surface.

5. The method as recited in claim 3 wherein the porous structure has the singular structure having a structural or material gradient.

6. A medical implant comprising:
a) a titanium substrate bonded to
b) a dynamic porous structure comprising a dynamic porous material comprising a shape memory material.

7. The implant as recited in claim 6 wherein the shape memory material is nitinol.

8. The implant as recited in claim 6 wherein the medical implant is expandable to fill gaps between the medical implant and adjacent bone when implanted into a patient.

9. The implant as recited in claim 6 wherein the dynamic porous structure is metal injection molded.

10. The implant as recited in claim 6 wherein the dynamic porous structure comprises a singular structure.

11. The implant as recited in claim 6 wherein the medical implant has a non-dynamic core and a dynamic surface.

12. The implant as recited in claim 6 wherein the dynamic porous structure comprises a dodecahedron cellular structure.

13. A porous orthopedic implant comprising:
a titanium alloy substrate; and
a porous portion including a mixture of a material blended with a coated pore former and a homogenizing agent, wherein the porous portion comprises a Nitinol alloy.

13. The porous article as recited in claim 13 wherein the porous portion includes pores having a diameter of about 50 microns to about 2,000 microns.

15. The porous article as recited in claim 13 wherein the porous portion is about 60% to about 85% porous.
